(19) United States
(12) Patent Application Publication
(10) Pub. No.: US 2026/0294333 A1
(43) Pub. Date: Oct. 1, 2026

(54) METHODS AND SYSTEMS FOR CLASSIFYING SPEECH DATA

(71) Applicant: Peter Cohen Foundation dba EverythingALS, Seattle, WA (US)

(72) Inventors: Julián PELLER, Buenos Aires (AR); Indu Navar BINGHAM, Los Altos, CA (US); Stephanie HENZE, San Mateo, CA (US)

(73) Assignee: Peter Cohen Foundation dba EverythingALS, Seattle, WA (US)

(21) Appl. No.: 19/629,918

(22) Filed: Mar. 26, 2026

Related U.S. Application Data
(60) Provisional application No. 63/778,697, filed on Mar. 27, 2025.

(51) Int. Cl. A61B 5/00; G10L 15/183

(52) U.S. Cl. CPC A61B 5/4803; A61B 5/7267; G10L 15/183

(57) ABSTRACT

A non-transitory processor-readable medium stores instructions that, when executed by a processor, cause the processor to receive and provide first speech data as input to a context window of a first machine learning model to generate a first assessment metric value associated with a first assessment aspect from a plurality of assessment aspects. The instructions further cause the processor to receive second speech data. The second speech data and data from the context window of the first machine learning model are provided to a context window of a second machine learning model to generate a second assessment metric value associated with a second assessment aspect from the plurality of assessment aspects. An assessment outcome is determined based on the first assessment metric value and the second assessment metric value, and the instructions cause the processor to cause display at a user compute device of an indication of the assessment outcome.

### CROSS REFERENCE TO RELATED APPLICATIONS

This application claims priority to and the benefit of U.S. Provisional Application No. 63/778,697, filed Mar. 27, 2025, and titled “METHODS AND SYSTEMS FOR CLASSIFYING SPEECH DATA,” which is incorporated herein by reference in its entirety. This application is related to U.S. Nonprovisional application Ser. No. 19/170,611, filed Apr. 4, 2025, and titled “METHODS AND SYSTEMS FOR USING MACHINE LEARNING MODELS TO DETECT ANOMALIES AND PREDICT TRENDS IN SPEECH DATA,” which is incorporated herein by reference in its entirety.

### BACKGROUND

Some embodiments described herein relate to methods and systems for analyzing speech data and/or text data to facilitate an assessment of a user's condition.

### SUMMARY

In some embodiments, a non-transitory processor-readable medium stores instructions that, when executed by a processor, cause the processor to receive first speech data and provide the first speech data as input to a context window of a first machine learning model to generate a first assessment metric value associated with a first assessment aspect from a plurality of assessment aspects. The instructions further cause the processor to receive second speech data. The second speech data and data from the context window of the first machine learning model are provided to a context window of a second machine learning model to generate a second assessment metric value associated with a second assessment aspect from the plurality of assessment aspects. An assessment outcome is determined based on the first assessment metric value and the second assessment metric value, and the instructions cause the processor to cause display at a user compute device of an indication of the assessment outcome.

In some embodiments, a method includes causing, via a processor, a first question associated with a first assessment aspect from a plurality of assessment aspects to be presented to a user via a user compute device. After causing the first question to be presented, the method includes receiving, at the processor, first speech data that represents a response of the user to the first question. Additionally, the method includes providing, via the processor, the first speech data as input to a context window of a first machine learning model to generate a first assessment metric value associated with the first assessment aspect. A second question associated with a second assessment aspect from the plurality of assessment aspects is presented to the user via the user compute device, and after causing the second question to be presented, the method includes receiving, at the processor, second speech data that represents a response of the user to the second question. The second speech data and data from the context window of the first machine learning model is provided as input via the processor to a context window of a second machine learning model to generate a second assessment metric value associated with the second assessment aspect. An assessment outcome is determined via the processor and based on the first assessment metric value and the second assessment metric value, and an indication of the assessment outcome is presented to the user via the user compute device.

In some embodiments, a non-transitory, processor-readable medium stores instructions that, when executed by a processor, cause the processor to provide first speech data associated with a user as input to a context window of a first machine learning model to generate a first assessment metric value associated with a first assessment aspect from a plurality of assessment aspects. The instructions further cause the processor to provide (1) second speech data associated with the user and (2) data from the context window of the first machine learning model to a context window of a second machine learning model to generate a second assessment metric value associated with a second assessment aspect from the plurality of assessment aspects. A data structure is received, representing at least one of a population atemporal condition, a subject temporal condition, or a condition comparison through time, and the instructions cause the processor to at least one of (1) provide the data structure as input to the context window of the first machine learning model to generate a modified first assessment metric value, or (2) provide the data structure as input to the context window of the second machine learning model to generate a modified second assessment metric value. An assessment outcome is determined based on at least one of the first assessment metric value, the second assessment metric value, the modified first assessment metric value, or the modified second assessment metric value, and the instructions further cause the processor to cause display at a user compute device of an indication of the assessment outcome.

### BRIEF DESCRIPTION OF THE DRAWINGS

FIG. 1 is a schematic diagram of an assessment system, according to an embodiment.

FIG. 2 is a schematic diagram of a compute device included in an assessment system, according to an embodiment.

FIG. 3 is a schematic diagram of assessment components included in an assessment system, according to an embodiment.

FIG. 4 is a schematic diagram of a plurality of machine learning models included in an assessment system, according to an embodiment.

FIGS. 5A-5E depict an example of a graphical user interface configured to facilitate a user assessment, according to an embodiment.

FIG. 6 is a schematic diagram of a voice processing pipeline, according to an embodiment.

FIG. 7 is a flowchart showing a method of using an assessment system to determine an assessment outcome, according to an embodiment.

FIG. 8 is a flowchart showing a method for determining an assessment outcome based on a first assessment metric value and a second assessment metric value, according to an embodiment.

FIG. 9 is a flowchart showing a method for generating modified assessment values, according to an embodiment.

### DETAILED DESCRIPTION

Neurodegenerative and/or motor neuron diseases, such as Amyotrophic Lateral Sclerosis (ALS), Huntington's disease, Parkinson's disease, Alzheimer's disease, and/or the like, can cause progressive muscle weakness as a result of, for example, bulbar dysfunction. In some instances, progressive bulbar dysfunction can be marked by progressive complex dysarthria, which can lead to social isolation, reduced quality of life, etc. Dysarthria can be caused by dysfunction in any one or more speech subsystems (e.g., articulatory, resonatory, phonatory, respiratory, etc.). Complex disorders, such as ALS, can cause these one or more speech subsystems to decline at different rates, and compensatory mechanisms can further complicate patterns of dysarthria. Some methods of quantifying disease progression, such as the ALS Functional Rating Scale-Revised (ALSFRS-R), are typically completed and/or facilitated by a health professional (e.g., a doctor), which can be costly and/or logistically burdensome. The ALSFRS-R Self-Entry (ALSFRS-RSE) (e.g., a self-administered ALSFRS-R) can rely on patient and/or caregiver self-awareness, affecting reliability of the assessment. As a result, these methods can introduce subjective bias when assessing the progression of dysarthria. More specifically, these methods typically use patient recorded outcomes, and a patient's lack of self-awareness can affect accuracy. Thus, there is a need for improved methods and systems for quantifying dysarthria and predicting progression of symptoms associated with neurodegenerative and/or motor neuron diseases.

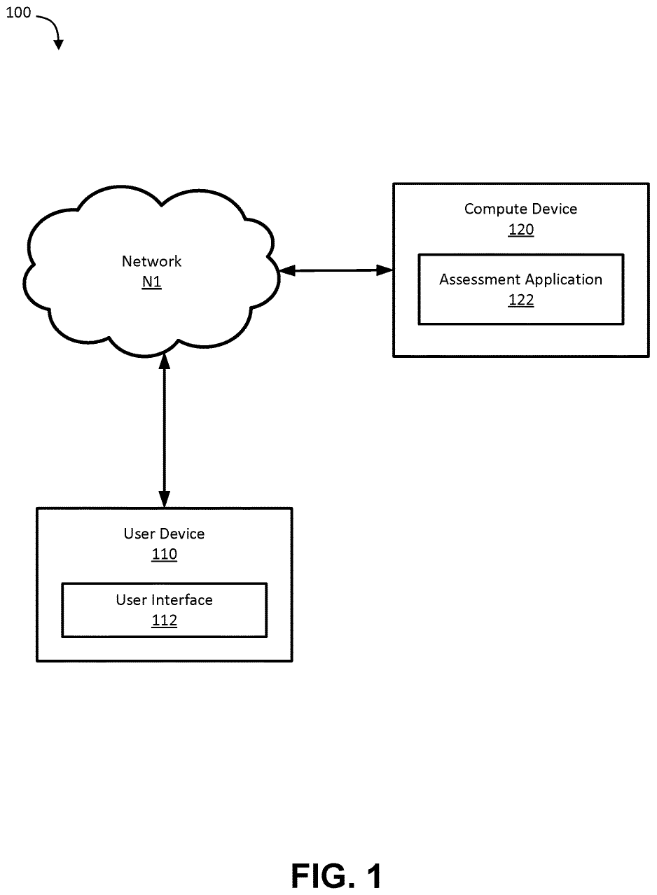

FIG. 1 is a schematic diagram of an assessment system 100 configured to facilitate an assessment of a user, according to an embodiment. The assessment system 100 includes a user device 110, a compute device 120, and a network N1. The assessment system 100 can include alternative configurations, and various steps and/or functions of the processes described below can be shared among the various devices of the assessment system 100 or can be assigned to specific devices (e.g., the user device 110, the compute device 120, and/or the like). For example, in some configurations, a user can provide inputs directly to the compute device 120 rather than via the user device 110, as described herein.

In some embodiments, the user device 110 and/or the compute device 120 can include any suitable hardware-based computing devices and/or multimedia devices, such as, for example, a server, a desktop compute device, a smartphone, a tablet, a wearable device, a laptop and/or the like. In some implementations, the user device 110 and/or the compute device 120 can be implemented at an edge node or other remote computing facility. In some implementations, each of the user device 110 and/or compute device 120 can be a data center or other control facility configured to run and/or execute a distributed computing system and can communicate with other compute devices.

In some implementations, the user device 110 can include a peripheral device(s) (not shown in FIG. 1) configured to receive an input from a user. For example, the user device 110 can include (1) a keyboard and/or other device that the user can use to generate text data, (2) a microphone that the user can use to record and/or generate audio (e.g., speech) data, (3) a speaker to audibly convey generated data to the user, and/or (4) an imaging device that can be used to generate image data and/or video data, to be analyzed by the compute device 120. The peripheral device(s) can further include a prosthetic device configured to, for example, capture and translate eye movement to text data (e.g., if the user is an advanced patient and/or has severe symptoms). The user device 110 and/or the compute device 120 can transcribe, interpret, and/or analyze the inputted data using an assessment application 122 (described herein). Moreover, in some implementations, the user device 110 and/or the compute device 120 can augment an assessment by using facial recognition techniques (e.g., applied to image data and/or video data from the imaging device) to infer a user's condition (e.g., an assessment metric value, a disease classification/diagnosis, a disease quantification/prognosis, etc.).

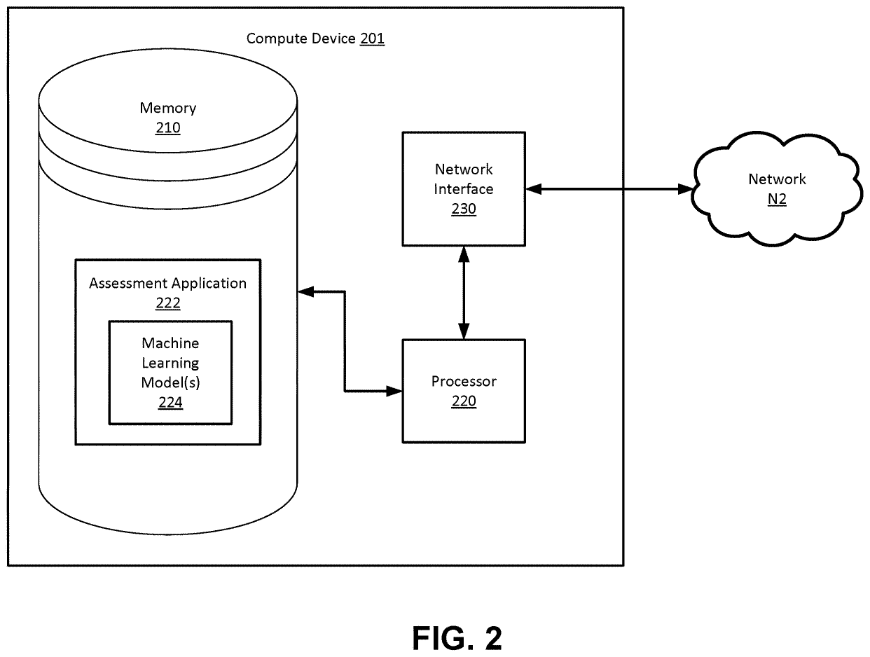

The compute device 120 can be configured to generate output data (e.g., response data, follow-up data, metric value data, etc.) associated with an assessment and based on input data received from the user (e.g., via the user device 110). To generate the output data, the compute device 120 can be configured to execute (e.g., via a processor) an assessment application 122, which can be functionally and/or structurally equivalent to the assessment application 222 of FIG. 2, described herein. The assessment application 122 can be implemented via software and/or hardware and can use one or more machine learning models to facilitate a conversation flow and/or generate one or more predictions based on input data (e.g., speech data, text data, etc.) received from the user device 110. Specifically, as described herein (e.g., in relation to FIGS. 2-4), the assessment application 122 can execute the one or more machine learning models to generate query data, follow up data, response data, etc., to guide a user through an assessment (e.g., an ALSFRS-R assessment, etc.). The assessment application 122 can be further configured to calculate a speech metric value and/or a listener effort metric value based on audio and/or transcription data and/or text data and/or video data. In some implementations, the assessment application 122 can be configured to aggregate input data over time to generate prior trend data and/or predict future trend data.

The user device 110 can be networked and/or communicatively coupled to the compute device 120, via the network N1, directly using wired connections and/or wireless connections. The network N1 can include various configurations and protocols, including, for example, short range communication protocols, Bluetooth®, Bluetooth® LE, the Internet, World Wide Web, intranets, virtual private networks, wide area networks, local networks, private networks using communication protocols proprietary to one or more companies, Ethernet, WiFi and/or HTTP, cellular data networks, satellite networks, free space optical networks and/or various combinations of the foregoing. Such communication can be facilitated by any device capable of transmitting data to and from other compute devices, such as a modem(s) and/or a wireless interface(s).

In some implementations, although not shown in FIG. 1, the assessment system 100 can include a plurality of user devices 110 and/or compute devices 120. For example, in some implementations, the assessment system 100 can include a plurality of user devices 110, where each user device 110 can be associated with a different user from a plurality of users. In some implementations, a plurality of user devices 110 can be associated with a single user, where each user device 110 can be associated with, for example, a different input modality (e.g., text input, audio input, video input, etc.).

FIG. 2 is a schematic diagram of a compute device 201 that can be included in an assessment system, according to an embodiment. The compute device 201 can be structurally and/or functionally similar to, for example, the compute device 120 of the assessment system 100 shown in FIG. 1. The compute device 201 can be a hardware-based computing device, a multimedia device, or a cloud-based device such as, for example, a computer device, a server, a desktop compute device, a laptop, a smartphone, a tablet, a wearable device, a remote computing infrastructure, and/or the like. The compute device 201 includes a memory 210, a processor 220, and a network interface 230 operably coupled to a network N2.

The processor 220 can be, for example, a hardware based integrated circuit (IC), or any other suitable processing device configured to run and/or execute a set of instructions or code (e.g., stored in memory 210). For example, the processor 220 can be a general-purpose processor, a central processing unit (CPU), an accelerated processing unit (APU), an application specific integrated circuit (ASIC), a field programmable gate array (FPGA), a programmable logic array (PLA), a complex programmable logic device (CPLD), a graphics processing unit (GPU), a programmable logic controller (PLC), a remote cluster of one or more processors associated with a cloud-based computing infrastructure and/or the like. The processor 220 is operatively coupled to the memory 210 (described herein). In some embodiments, for example, the processor 220 can be coupled to the memory 210 through a system bus (for example, address bus, data bus and/or control bus).

The memory 210 can be, for example, a random-access memory (RAM), a memory buffer, a hard drive, a read-only memory (ROM), an erasable programmable read-only memory (EPROM), and/or the like. The memory 210 can store, for example, one or more software modules and/or code that can include instructions to cause the processor 220 to perform one or more processes, functions, and/or the like. In some implementations, the memory 210 can be a portable memory (e.g., a flash drive, a portable hard disk, and/or the like) that can be operatively coupled to the processor 220. In some instances, the memory can be remotely operatively coupled with the compute device 201, for example, via the network interface 230. For example, a remote database server can be operatively coupled to the compute device 201.

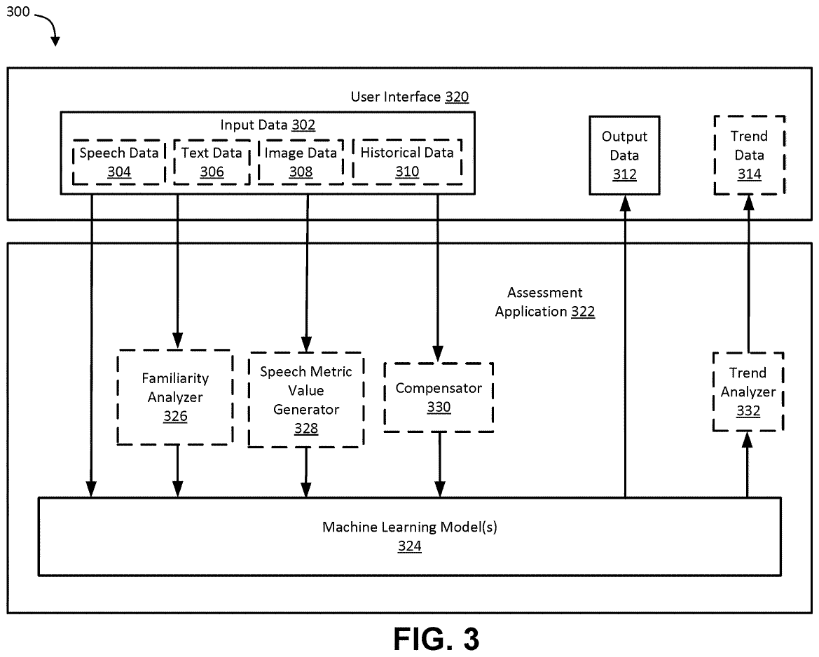

The memory 210 can store various instructions associated with processes, algorithms and/or data, including machine learning models (e.g., a neural network, a regression model, a speech recognition model, etc., as described herein). Memory 210 can further include any non-transitory computer-readable storage medium for storing data and/or software that is executable by processor 220, and/or any other medium which may be used to store information that may be accessed by processor 220 to control the operation of the compute device 201. For example, the memory 210 can store data associated with an assessment application 212. The assessment application 222 can be functionally and/or structurally similar to the assessment application 122 of FIG. 1 and/or the assessment application 322 of FIG. 3, described herein.

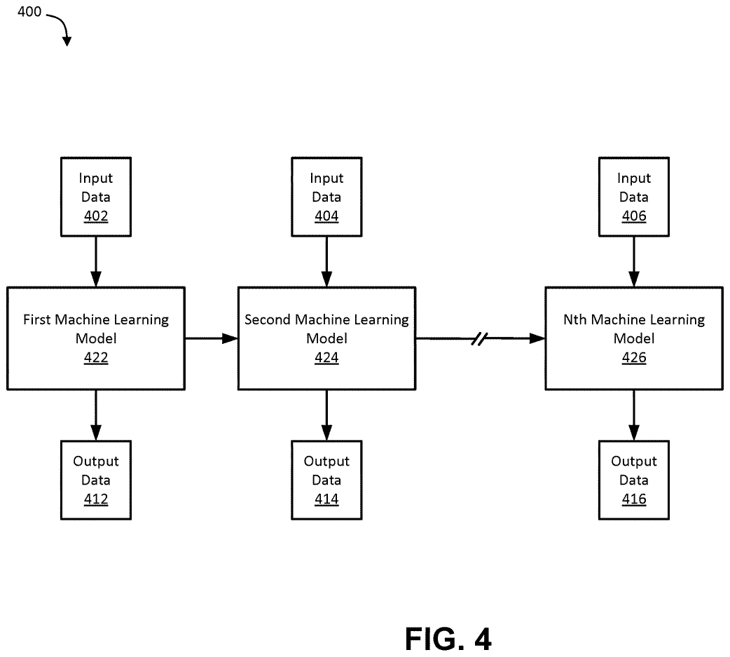

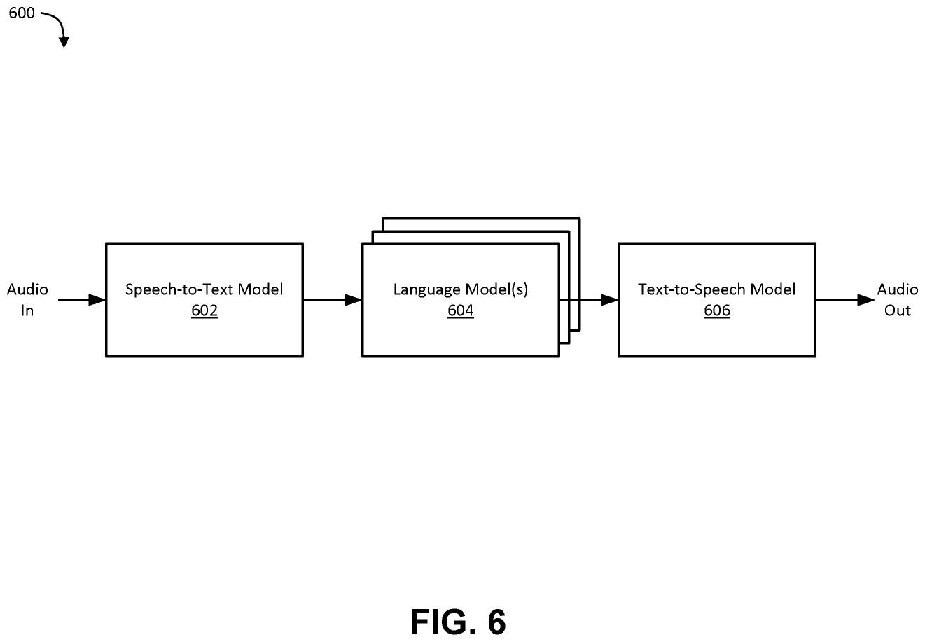

The assessment application 222 includes (or has access to) a machine learning model(s) 224, which can be functionally and/or structurally similar to the machine learning model(s) 324 of FIG. 3; the first, second, and nth machine learning models 422-426 of FIG. 4; and/or the language model(s) 604 of FIG. 6; described in further detail herein. Although FIG. 2 shows the machine learning model(s) 224 being stored within the memory 210, in some implementations, the machine learning model(s) 224 can be implemented on a remote compute device different from the compute device 201 and communicatively coupled to the compute device 201 via the network N2 (described below). For example, the machine learning model(s) 224 can be implemented by a third party compute device and accessed from the compute device 201 (e.g., using the assessment application 222) via an application programming interface (API). In response to receiving input data from a user, the assessment application 222 can cause the machine learning model(s) 224 to produce response data to guide the user through an assessment (e.g., a patient reported outcome), as described further herein.

The network interface 230 can be configured to connect to the network N2, which can be functionally and/or structurally similar to the network N1 of FIG. 1. For example, network N2 can use any of the wired and wireless short range communication protocols described above with respect to network N1 of FIG. 1.

In some instances, the compute device 201 can further include a display, an input device, and/or an output interface (not shown in FIG. 2). The display can be any display device by which the compute device 201 can output and/or display data. The input device can include a mouse, keyboard, touch screen, voice interface, and/or any other hand-held controller or device or interface via which a user may interact with the compute device 201. The output interface can include a bus, port, and/or other interfaces by which the compute device 201 may connect to and/or output data to other devices and/or peripherals, such as a speaker.

FIG. 3 is a schematic diagram of assessment components 300, according to an embodiment. The assessment components 300 can be associated with and/or executed by a compute device (e.g., a compute device that is structurally and/or functionally similar to the compute device 201 of FIG. 2 and/or the user and/or compute devices 110 and 120 of FIG. 1). In some instances, for example, the assessment components 300 can be software stored in memory 210 and configured to be executed via the processor 220 of FIG. 2. In some instances, for example, at least a portion of the assessment components 300 can be implemented in hardware. The assessment components include input data 302, an assessment application 322, output data 312, and (optionally) trend data 314. The input data 302 can be associated with a user interface 320, which can be structurally and/or functionally equivalent to the user interface 112 of FIG. 1. For example, the input data 302 can be received via and/or displayed on the user interface 320, as described herein. The assessment application 322 can be structurally and/or functionally similar to the assessment application 122 of FIG. 1 and/or the assessment application 222 of FIG. 2. The assessment application 322 includes a machine learning model(s) 324, which can be structurally and/or functionally similar to the machine learning model(s) 224; the first machine learning model 422, the second machine learning model 424, and/or the nth machine learning model 426 of FIG. 4; and/or the language model(s) 604 of FIG. 6; described in further detail herein. Optionally, the assessment components 300 can include a familiarity analyzer 326, a speech metric value generator 328, a compensator 330, and a trend analyzer 332.

The assessment application 322 can be configured to guide a user through an assessment to, for example, determine a patient reported outcome. In the context of ALS, a patient reported outcome can be associated with ALSFRS-R, the ALS Respiratory Symptom Scale (ARES), the Communicative Participation Item Bank (CPIB), the Rasch Overall ALS Disability Scale (ROADS), and/or the like. For Parkinson's Disease, a patient reported outcome can be associated with the Unified Parkinson's Disease Rating Scale (UPDRS) and/or the like. For Huntington's Disease, the patient reported outcome can be associated with the Unified Huntington's Disease Rating Scale (UHDRS) and/or the like, and for Alzheimer's Disease and/or dementia, the patient reported outcome can be associated with the Clinical Dementia Rating Scale (CDR) and/or another assessment for a patient reported outcome for a disease.

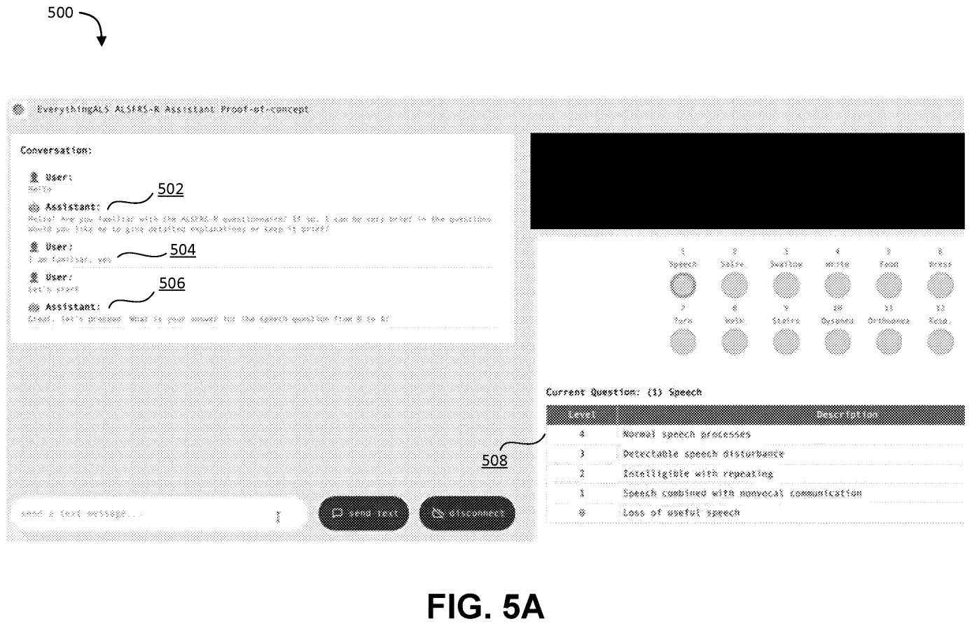

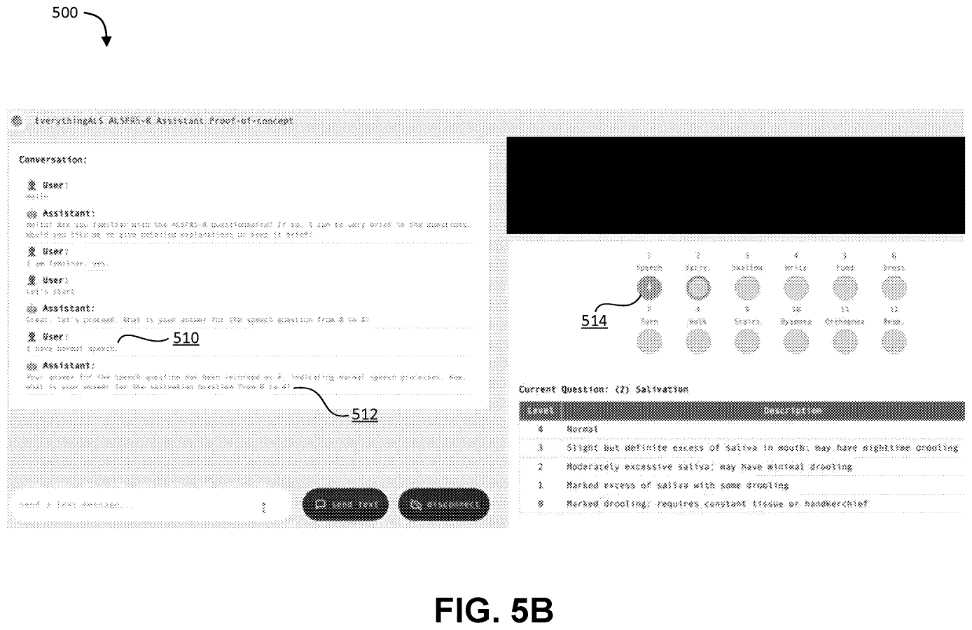

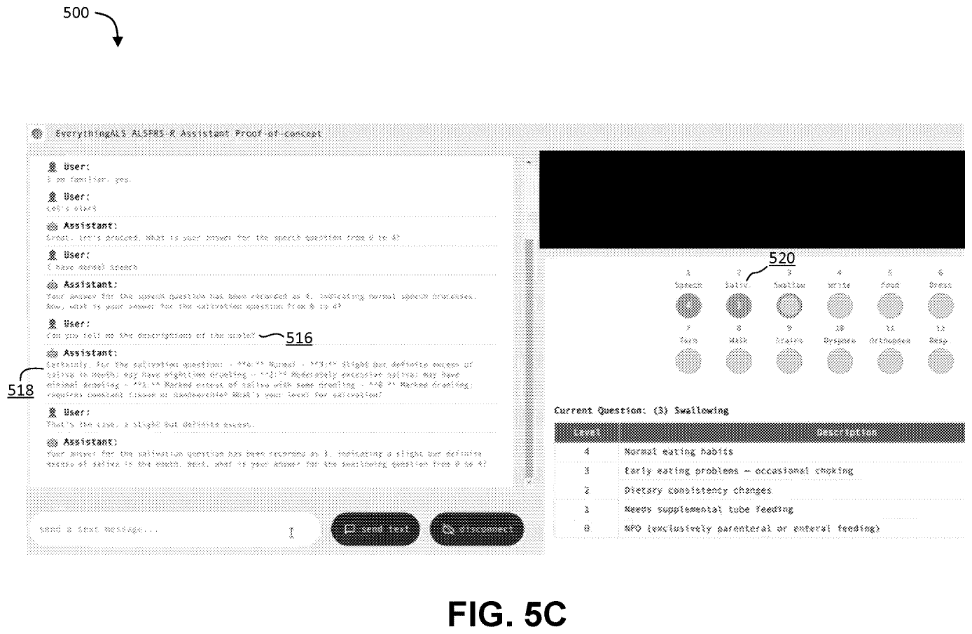

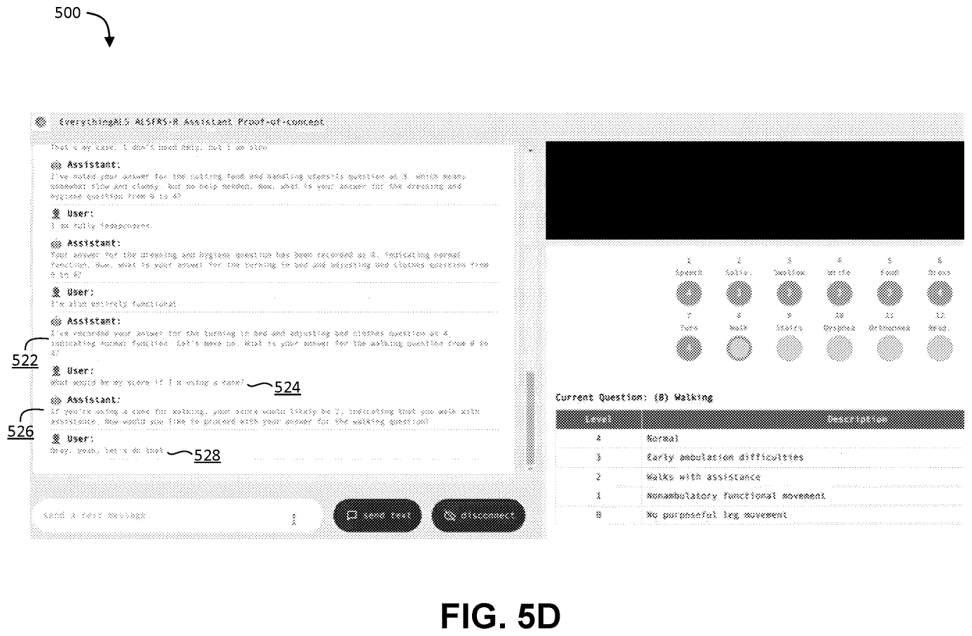

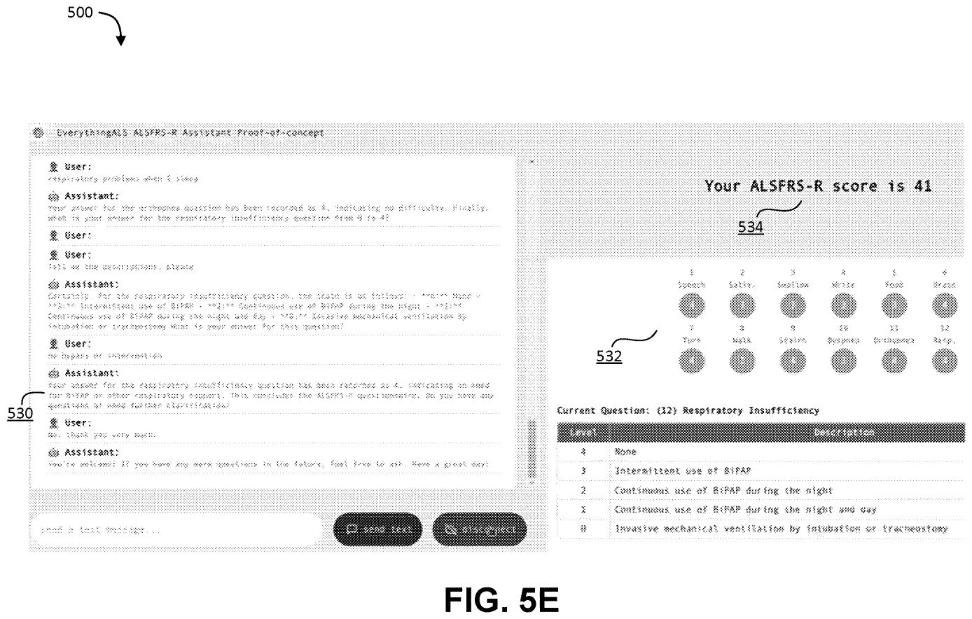

To guide the user through the assessment, the assessment application 322 can predict and/or identify a user's state based on the input data 302 provided by the user. More specifically, the assessment application 322 can generate output data 312 that represents a metric value(s) associated with a patient reported outcome, as illustrated further herein at least in relation to FIGS. 5A-5E.

The Revised ALS Functional Rating Scale (ALSFRS-R) is used herein to illustrate an example implementation of the assessment application 322. ALSFRS-R is a validated rating instrument (e.g., a questionnaire-based scale) for monitoring the progression of disability in patients with ALS. The ALSFRS-R measures 12 aspects of physical function, ranging from one's ability to swallow and use utensils to climbing stairs and breathing. Each function is scored from 4 (normal) to 0 (no ability), with a maximum total score of 48 and a minimum total score of 0. The assessment application 322 can be configured to educate the user and/or elicit responses from the user to provide a metric value for at least one aspect (e.g., each aspect) from the 12 aspects associated with the ALSFRS-R. As described further herein, the assessment application 322 can include (or have access to) a plurality of machine learning models (e.g., included in the machine learning model(s) 324), where each machine learning model from the plurality of machine learning models is associated with a different aspect from the 12 aspects associated with the ALSFRS-R. Alternatively, the machine learning model(s) 324 can include a machine learning model configured to facilitate a plurality of assessment aspects (e.g., each aspect of the assessment). Alternatively, the machine learning model(s) 324 can include a plurality of instances of a machine learning model that is configured (e.g., prompted) differently for different aspects of an assessment.

The input data 302 can include at least one of speech data 304 (e.g., audio data), text data 306, image data 308 (e.g., video data), and/or historical data 310, received from a user and/or another source (e.g., an electronic health record, a clinician, stored application data, etc.). In some instances, the user can be diagnosed with and/or exhibit symptoms associated with a neurodegenerative and/or motor neuron disease, such as ALS, Alzheimer's disease, Huntington's disease, Parkinson's disease, and/or the like. To generate the speech data 304 (e.g., audio data), the user can speak into a microphone to generate the audio data. In some implementations, the user can use the user interface 320 to capture the speech data 304. For example, the user interface 320 can be implemented by a compute device that is structurally and/or functionally equivalent to the user device 110. A user can initiate a recording function (e.g., by selecting a selectable element implemented by the user interface 320) via the user interface 320 to record audio via a microphone included in and/or operably coupled to the compute device. To generate the text data 306, the user can use a keyboard or another assistive device (e.g., an eye-controlled text entry device), operably coupled to the compute device. To generate the image data 308 (e.g., image data), a camera (e.g., a web cam) operably coupled to the compute device can record video of and/or take images of the user. For example, the camera can record a user's facial expressions as the user speaks, a user while eating, a user while putting a coat on, and/or the like.

The historical data 310 can be associated with a population level and/or a user level. In some instances, the historical data 310 can include clinical data. At a population level, the historical data 310 can indicate a state (e.g., diagnosis, prognosis, assessment metric value, etc.) that an individual from the population is typically (e.g., on average) assigned for a given condition (e.g., symptom, ability level, etc.). At a user level, the historical data 310 can represent a historical record (e.g., clinical history, prior observations, etc.) associated with the user (e.g., and not others from a population). The historical data 310 can be received by the compensator 330 to potentially modify and/or validate the output of the machine learning model(s) 324, as described further herein.

In some implementations, the input data 302 can include image data 308, which can depict a user's face (e.g., while speaking, chewing, and/or etc.) and/or include video data depicting a user's movement (e.g., while the user walks up the stairs, while the user eats, while the user puts on a coat, and/or etc.). The assessment application 322 can be configured to predict, for example, a quantification (e.g., a prognosis, an assessment metric value, etc.) and/or progression rate (e.g., assessment metric trend) based on the image data 308 (e.g., in addition to other data from the input data 302). In some instances, the assessment application 322 can be configured to analyze a user's visual attributes and/or movements (e.g., facial expression, facial droop, twitching, shaking, etc.) to influence an assessment metric, to generate a baseline prediction to compare an assessment metric value to, and/or to produce a follow-up question posed to the user, as described further herein. The image data can be provided as an input to the machine learning model(s) 324, which can include a convolutional neural network (CNN), a multimodal large language model (multimodal LLM), a vision language model (VLM), and/or a similarly suited model for processing image and/or video data to produce the output data 312 (e.g., including assessment metric data), as described further herein. A CNN can be trained to generate, at least in part, an assessment metric and/or a progression rate based on image data and/or video data depicting other users with known assessment metrics (e.g., as determined by a health professional, such as a speech-language pathologist). In some implementations, the assessment application 322 can be configured to perform facial landmark analysis to identify features in the image data 308, and these features can be used (e.g., in addition to other input data 302, such as the speech data 304) to generate an assessment metric value, progression rate, and/or the like.

The user interface 320 can further cause display and/or output of information to the user via a display operably coupled to the compute device. Such information can be represented by the output data 312 and can include, for example, text data, audio data, image data, and/or the like. The output data can convey (e.g., depict, announce, etc.), for example, a response to a user's query, an indication of an assessment metric value, a description and/or clarification of an aspect of an assessment, a trend, etc., as described further herein.

The machine learning model(s) 324 can be configured to facilitate audio, text (e.g., chat), and/or video interaction with a user undergoing an assessment (e.g., a user completing a patient reported outcome). As described further below at least in relation to FIG. 4, the machine learning model(s) 324 can include a plurality of machine learning models, such as a plurality of transformer-based models (e.g., a plurality of large language models (LLMs)). Each machine learning model from the plurality of machine learning models can be associated with a different aspect (e.g., question) of an assessment. For example, each machine learning model can be configured (e.g., trained, prompted, etc.) to produce an output for a respective question from a plurality of questions associated with the assessment. An output and/or data from a context window of a first machine learning model can be provided as input to and/or to a context window of a second machine learning model, such that data for a first question associated with the first machine learning model can inform a second question associated with the second machine learning model. This multi-model configuration can additionally permit the first machine learning model and the second machine learning model (and/or any other machine learning model from the plurality of machine learning models) to adapt and/or evolve over time based on interactions with the user, as described further herein. Moreover, the multi-model configuration can facilitate cohesive and/or congruent outputs among the plurality of machine learning models, as described further herein.

The output data 312 can represent an output(s) of the machine learning model(s) 324 while the machine learning model(s) 324 conducts a conversation with the user. For example, the output data 312 can include each assessment metric value associated with each aspect of the assessment and produced by each respective machine learning model from the machine learning model(s) 324. Alternatively or in addition, the assessment application 322 can aggregate each assessment metric value associated with each aspect to produce an overall assessment metric value (e.g., similar to the overall assessment metric value 534 of FIG. 5E, described further herein), which the assessment application 322 can include in the output data 312. In some implementations, as described further below, the assessment application 322 can be configured to include in the output data 312 a familiarity metric and/or clarification data produced by the familiarity analyzer 326, a speech metric(s) and/or listener effort metric generated by the speech metric generator 328, and/or compensation data produced by the compensator 330. Alternatively or in addition, the outputs of at least some of the foregoing components of the assessment application 322 can be stored (e.g., in a memory functionally and/or structurally similar to the memory 210 of FIG. 2) for subsequent analysis (e.g., by a clinician and/or the like).

As described further below, in addition to (or instead of) the input data 302 (e.g., at least one of the speech data 304, the text data 306, the image data 308, and/or the historical data 310), in some implementations, the machine learning model(s) 324 can receive, as input, an output(s) from the familiarity analyzer 326, the speech metric generator 328, and/or the compensator 330.

Optionally, the familiarity analyzer 326 can cause the machine learning model(s) 324 to adapt the outputs of the machine learning model(s) 324 based on the user's inferred familiarity with an aspect(s) of the assessment. For example, the familiarity analyzer can include a machine learning model (e.g., a transformer-based model, such as a large language model (LLM), a speech analysis model, such as a transcription model, and/or the like) configured to identify (1) a question posed by the user (e.g., based on the user's intonation within recorded speech), (2) a user's uncertainty and/or hesitancy (e.g., based on a length and/or number of pauses within the user's recorded speech), and/or a word(s) that indicates the user's unfamiliarity with an aspect of the assessment (e.g., “what,” “how,” “describe,” “can you,” etc.). In some implementations, although not shown in FIG. 3, the familiarity analyzer 326 can be implemented by at least one machine learning model from the machine learning model(s) 324. For example, an agent associated with an assessment aspect and from the machine learning model(s) 324 can be configured to perform the functions of the familiarity analyzer.

In some instances, the familiarity analyzer 326 can be implemented by an application programming interface (API) configured to facilitate real-time communication (RTC). For example, the familiarity analyzer 326 can be implemented using WebRTC and/or the like, such that the familiarity analyzer 326 can stream data to and/or from a user compute device and/or the user interface 320 to interrupt and/or provide clarification during a conversation between the assessment application 322 and the user. Examples of clarifications that can be facilitated by the familiarity analyzer 326 are described further herein at least in relation to FIGS. 5A-5E.

In some instances, the familiarity analyzer 326 and/or the machine learning model(s) 324 can generate a question posed to the user to ask explicitly about the user's familiarity with an assessment question. Alternatively or in addition, the familiarity analyzer 326 and/or the machine learning model(s) 324 (e.g., an agent) can generate a familiarity label(s) (e.g., in response to the user uttering certain words indicative of familiarity), which the assessment application 322 can provide to other machine learning models from the machine learning model(s) 324 (e.g., as described further at least in relation to FIG. 4). In some implementations, the user can, via the user interface, modify a familiarity level assigned by the assessment application 322.

The speech metric generator 328 optionally included in the assessment components 300 can be configured to generate, based on the speech data 304 (e.g., audio data) included in the input data 302, at least one of transcription data, timestamp data associated with the transcription data, and/or confidence data associated with the transcription data. The speech metric generator 328 can include a speech recognition model, a speech-to-text model, an encoder-decoder model, a transformer model and/or the like. Example implementations of the machine learning model included in the speech metric generator 328 can include a Whisper model and/or a model similarly suited for transcribing audio data. The transcription data can include a set of words transcribed from the audio data. The timestamp data can indicate a relative time that a transcribed word was captured in the audio data as compared to other transcribed words captured in the audio data. For example, the user can utter a first word followed by a second word while the assessment application 322 records the speech data 304. As a result, the speech metric generator 328 can generate a first timestamp for the first word and, for the second word, a second timestamp that indicates a later time than the first timestamp. The difference between the second timestamp and the first timestamp can indicate the time period between when the user spoke the first word and the second word. In another implementation, the speech metric generator 328 can generate two or more timestamps for each word included in the transcription data. For example, for a given word, a first timestamp can be associated with the beginning of the word (e.g., a time associated with when a user begins to speak the word) and a second timestamp can be associated with the end of the word (e.g., a time associated with when the user is finished speaking the word). The first and second timestamps can be used to determine a time interval between words included in the transcription data, the duration of each word, and/or metrics associated with, for example, speech duration and/or rate.

The confidence data optionally generated by the speech metric generator 328 can include, for example, a set of confidence metrics (e.g., confidence scores), and each confidence metric from the set of confidence metrics can be associated with a different word from the set of words included in the transcription data. The speech metric generator 328 can generate a confidence metric for a transcribed word to indicate a level of certainty in the accuracy of the transcription for that word. For example, if the user clearly annunciates a word, and that word is represented clearly within the audio data, the confidence metric for that word can be high. Alternatively, if the user, for example, mumbles or slurs a word, or the word is muffled within the audio data, the speech metric generator 328 can interpret (e.g., classify) the word with less accuracy, leading to a lower confidence metric for that word. In some implementations, a plurality of confidence metrics for a plurality of words included in the transcription data can be combined (e.g., averaged, represented as a standard deviation, etc.) to produce a collective (e.g., average) confidence metric for the transcription data. In some implementations, multiple collective confidence metrics (e.g., average, mode, standard deviation, median, etc.) can be calculated based on the plurality of confidence metrics for the plurality of words.

A layer and/or model of the speech metric generator 328 can be configured to receive the transcription data (and/or data associated with the transcription data), and the timestamp data generated by another layer and/or model of the speech metric generator 328 and, in response, generate at least one speech metric. For example, based on the number of words included in the transcription data (and/or the number of syllables, etc., included in the transcript data) and the timestamp data for each word, the speech metric generator 328 can generate at least one of a speaking rate (e.g., an average number of words spoken by the user per minute and/or other unit of time), an articulation rate (e.g., an average number of syllables spoken by the user per second and/or other unit of time), and/or any other measure of speaking pace.

The speech metric generator 328 can be further configured to predict a listener effort metric based on (1) the confidence metric(s) (e.g., the collective confidence metric(s), the set of confidence metrics for the set of words included in the transcription data and/or the average confidence metric for the transcription data), (2) the speech metric(s) generated by the speech metric generator 328, and/or (3) additional inputs (e.g., the speech data 304 and/or other data from the input data 302, the transcription data, etc.). A listener effort metric can include, for example, a relative measure (e.g., on a scale of 0-100) of a listener's allocation of their mental resources to resolve ambiguity and/or errors included in the user's spoken dialogue. For example, in some implementations, a higher listener effort metric can indicate an increased level of unintelligibility (e.g., speech that is harder to understand as would be perceived by, for example, a speech-language pathologist) associated with the user's speech data 304.

In some implementations, the speech metric generator 328, by receiving the speech data 304 as input, can be configured to identify acoustical features of the speech data 304, and these acoustical features can be provided as input to another layer and/or model of the speech metric generator 328 to predict the listener effort metric. Examples of acoustical features can include a sound envelope metric (e.g., variations in sound envelope), a fundamental frequency, a jitter metric, a shimmer metric, a pitch metric, a formants metric (e.g., variations in the second vocal tract formant F2 computed, for example, using Parselmouth library), a formants variation (e.g., variant and/or derivate) metric, a harmonic-to-noise ratio (HNR), a Wiener entropy (or a similar entropy) metric, a Cepstral peak prominence (CPP) metric, and/or other acoustical features that can be indicative of bulbar decline.

In some implementations, at least some of the foregoing acoustical features can be determined based on a spectrogram, which can represent a frequency decomposition of the speech data 304. The spectrogram can be generated as an output of the speech metric generator 328 or another component of the assessment application 322, such as a machine learning model from the machine learning model(s) 324. More specifically, the speech metric generator 328 and/or the machine learning model(s) 324 can include (or have access to) a convolutional neural network (CNN), a recurrent neural network (RNN), and/or the like, configured to receive spectrogram data as input to generate the acoustical feature(s), such as pitch, a formants metric(s), CPP, HNR, etc.

In some implementations, an acoustical feature(s) (e.g., one or more of the foregoing acoustical features) can be selected from a plurality of acoustical features, and the selected acoustical feature(s) (and not, for example, any remaining acoustical features from the plurality of acoustical features) can be provided as an input to a model and/or model layer (e.g., of the speech metric generator 328 and/or the machine learning model(s) 324) to predict the listener effort metric. In some implementations, the acoustical feature(s) can be selected from the plurality of acoustical features as part of training the model and/or the model layer. To select an acoustical feature from the plurality of acoustical features, an average value for each acoustical feature from the plurality of features can be determined for a given session(s) (e.g., for an initial assessment(s)) completed by a given user. After the given user has completed a plurality of sessions over a period of time, for each acoustical feature, average values can be determined for each session from the plurality of sessions. Also, for each acoustical feature, a trajectory can be generated for the given user based on the plurality of average values associated with the plurality of sessions, and linear regression can be used determine slopes of the resulting trajectories associated with the plurality of acoustical features. An acoustical feature from the plurality of acoustical features can be determined to be significant if, for example, at least a threshold percentage (e.g., 5%) of users (e.g., a group of users that includes the given user, such as a group of patients) are associated with trajectories that are associated with that acoustical feature and that have slopes (e.g., an absolute value of the slope) that are greater than those observed in a control group. An acoustical feature that is determined to be significant can be calculated for another user (e.g., a user not included in the aforementioned group of users) and provided as an input to the model and/or model layer to predict a listener effort metric for that other user.

In some implementations, the speech metric generator 328 can be further configured to generate, based on the confidence metric(s), the speech metric(s), and/or additional inputs (e.g., the audio date included in the input data 302, the transcription data, etc.), an overall dysarthria severity metric, a voice strain metric, a consistency metric (e.g., based on a word and/or phrase repeated multiple times by the user), an intelligibility metric, an articulatory precision metric, a dysphonia severity metric, a hypernasality metric, a breath support metric, a prosody metric, and/or the like. In some implementations, the speaking generator 328 can be further configured to generate a disease quantification metric (e.g., a prognosis, a life expectancy metric, and/or the like) based on the listener effort metric and/or the like.

In some implementations, the speaking generator 328 can include a Least Absolute Shrinkage and Selection Operator (Lasso) regression model and/or a model similarly suited for predicting and identifying relevant features in audio data to determine the listener effort metric. In some implementations, the speech metric generator 328 can include a neural network (e.g., a deep learning model configured to operate on audio data), a random forest model, a nearest neighbors model, and/or the like. In some implementations, the speech metric generator 328 can include a convolutional neural network (CNN) and/or a CNN layer that can generate the listener effort metric and/or an acoustical feature(s) (e.g., pitch, formants, CPP, HNR, etc.), based on a spectrogram (which can be generated by the speech metric generator 328 and/or another component of the assessment application 322, as described above). The listener effort metric and/or an acoustical feature(s) can then be used by the speech metric generator 328 (e.g., by a machine learning model that is included in the speech metric generator 328 and separate from the CNN) to generate the listener effort metric.

The speech metric generator 328 can be trained using training data generated by one or more health professionals (e.g., speech-language pathologists). For example, recorded speech data from a randomly selected participant can be presented to the health professional, and the health professional can assign a listener effort metric based on the health professional's experience. In some implementations, the health professional can assign the listener effort metric using a graphical user interface (GUI) (e.g., a GUI functionally and/or structurally similar to the GUI 500 of FIGS. 5A-5E, described herein).

The speech metric(s), listener effort metric, and/or any other metric calculated by the speech metric generator 328 and/or any other output of the speech metric generator 328 described above can be provided as input to at least one machine learning model from the machine learning model(s) 324 to inform generation of the output data 312. More specifically, based on the speech metric(s) and/or listener effort metric, the machine learning model(s) 324 can suggest and/or assign an assessment metric value associated with an assessment and/or indicate an observation. For example, based on a user's speaking rate being lower than a threshold value (e.g., the users historical average speaking rate and/or a fixed/predetermined threshold value), the machine learning model(s) 324 can infer whether a user's subjective response (e.g., user-defined assessment metric value) to an assessment question is consistent with the user's speaking rate. The machine learning model(s) 324 can be further configured to communicate to the user an inconsistency between the speaking rate and the subjective response, automatically (e.g., without human intervention) modify the user's subjective response based on the inconsistency to produce a modified assessment metric value (e.g., stored in addition to the user's self-defined assessment metric value), and/or store an indication of the inconsistency and/or the speaking rate for subsequent review (e.g., by a health professional and/or the like).

The compensator 330 can be configured to produce modified assessment data based on the input data 302. More specifically, the compensator 330 can modify or confirm a user's subjective assessment based on prior objective data from the user and/or a population that includes and/or is representative of the user. For example, in some implementations, the compensator 330 can receive as input a user-defined metric value for a first aspect of an assessment and detect an inconsistency with a user-defined metric value for a second aspect of the assessment. Alternatively or in addition, the compensator 330 can be configured to receive as input an indication of the user's condition (e.g., a clinical record, user statement, etc., indicating a symptom, diagnosis, prognosis, etc.) and can detect an inconsistency (or consistency) with a user-defined metric value for an aspect of the assessment. Alternatively, or in addition, the compensator 330 can be configured to receive, as input data, data representing an expected (e.g., average) assessment metric value across a population for a given condition(s) (e.g., symptom(s), diagnosis/diagnoses, prognosis/prognoses, etc.). Alternatively or in addition, the compensator 330 can receive audio data from the user (e.g., an audio recording of the user answering a question) and determine that the speaking quality captured in the audio data is inconsistent (or consistent) with a user-defined metric value for speaking quality (or another aspect of an assessment). In some implementations, the compensator 330 can cause the machine learning model(s) 324 (or another component of the assessment application 322) to modify or verify the user's assessment of the user's own condition. Alternatively, or in addition, the compensator 330 can cause an objective (e.g., compensated) assessment metric value, in addition to the user's subjective assessment metric value, to be stored (e.g., for subsequent analysis by a health professional). Examples of the compensator 330 in use are described further herein at least in relation to FIGS. 5A-5E.

To illustrate the compensator 330 in use, an example user can indicate that the user has substantially deteriorated speech as part of a first aspect of an assessment (e.g., ALSFRS-R). If the user then indicates that the user's swallowing has not deteriorated (e.g., by providing an answer of “4” for the swallowing aspect of ALSFRS-R), the compensator 330 can be configured to flag the inconsistency, producing a modified assessment metric value indicating a more likely state of the user's swallowing (e.g., a “2” for the swallowing aspect of ALSFRS-R). The assessment application 322 can cause the modified assessment metric value to be stored for comparison with the user-defined assessment metric value (e.g., to determine the user's level of bias). Alternatively, or in addition, the assessment application 322 can be configured to ask the user (e.g., questions generated via the machine learning model(s) 324) to confirm the user's reported swallowing ability. The assessment application 322 can be further configured to ask the user (e.g., questions generated via the machine learning model(s) 324) follow-up (e.g., more detailed) questions to clarify the user's swallowing ability.

In some instances, the historical data 310 can include a data structure(s) that can represent a population atemporal condition (e.g., a condition associated with an individual who is not the user), a subject/user temporal condition (e.g., a condition associated with a user at a time prior to a current execution of the assessment application), a comparison of conditions through time, etc. The compensator 330 can receive the data structure(s) as input to detect inconsistencies with a user-defined metric value for an assessment aspect.

In some implementations, the assessment application 322 can be configured to recognize, during a first conversation that is facilitated by a first machine learning model (e.g., a first agent model) from the machine learning model(s) 324, a user's speech and/or response pattern in the input data 302. In response, the assessment application 322 can execute a second machine learning model (e.g., a second agent model) from the machine learning model(s) 324 to carry on a second conversation to address the speech and/or response pattern. The second machine learning model can be configured to address a more specific topic (e.g., an issue that the speech and/or response pattern is indicative of), whereas the first machine learning model can be configured to address a more general topic (e.g., to facilitate an assessment aspect rather than address a more specific issue within the assessment aspect).

In some implementations, the compensator 330 can be configured to predict whether the user is prone to bias when self-assessing the user's condition. For example, the compensator 330 can determine an objective measure of the user's condition (e.g., based on a speech metric value determined by the speech metric value generator 328, the historical data 310, and/or the like, as described herein). The compensator 330 can then compare the objective measure to the user's self-defined, subjective measure to determine whether the user has a tendency to underestimate or overestimate the user's condition.

Optionally, the assessment application 322 can further include the trend analyzer 332, which can receive the input data 302 and/or the output data 312 as input to generate the trend data 314. For example, the trend analyzer 332 can include a model (e.g., a regression model, an extrapolation model, a machine learning model, etc.) that can be configured to predict a progression rate for a user's assessment metric value(s), symptom, prognosis, etc., based on a history of assessment metric values for the user over a time period and/or a history of the user's speech metric value(s) over time. Alternatively, or in addition, the trend analyzer 332 can be configured to predict a progression rate for the user based on a progression rate(s) for another (e.g., similarly situated) user(s), such as another user that had a similar assessment metric value(s) as the user. The progression rate can indicate a predicted change in the assessment metric value(s) (and/or speech metric value(s), prognosis, symptom(s), etc.) over a predefined time period (e.g., as measured from the time that a current assessment metric value was generated). The trend analyzer 332 can include, for example, a Mixture of Gaussian Processes (MoGP) model (and/or similarly suited unbiased machine learning model) to identify a user group (e.g., a cluster) associated with a similar progression rate as the user.

The assessment application 322 can be further configured to, in response to predicting the trend data 314, send a signal (e.g., to a compute device that is functionally and/or structurally similar to the user device 110 of FIG. 1) to cause display of a representation (e.g., a trendline) of the trend data 314, the current assessment metric value(s) for the user, and/or the at least one previous assessment metric value(s) for the user.

The trend analyzer 332 can further implement groupwise comparisons of respective listener effort progression rates for a plurality of users. These groupwise comparisons can be performed using, for example, a linear mixed model with random effects. These groupwise comparisons can include, for example, at least one of a) controls versus participants with ALS to determine if there was a difference in listener effort change over time; b) controls versus participants with ALS and, for example a score of 4 on ALSFRS-RSE Q1 throughout their participation, to determine whether any change was seen in people found to have no change in speech by self-report; c) participants with bulbar-onset ALS versus non-bulbar-onset ALS to determine if the slopes of decline differ based on site of onset; d) participants with bulbar-onset ALS versus non-bulbar-onset ALS excluding participants with normal listener effort at the outset of the study, to determine whether, once bulbar symptoms have begun, the progression rate varies depending upon whether onset was bulbar or non-bulbar.

FIG. 4 is a schematic diagram of a plurality of machine learning model components 400 included in (or accessible by) an assessment system, according to an embodiment. The assessment system can be functionally and/or structurally similar to the assessment system 100 of FIG. 1. The machine learning model components 400 can be associated with a compute device (e.g., a compute device that is structurally and/or functionally similar to the compute device 201 of FIG. 2 and/or the user device 110 and/or compute device 120 of FIG. 1). In some instances, the assessment components 300 can be software stored in memory 210 and configured to be executed via the processor 220 of FIG. 2. Alternatively, or in addition, at least a portion of the machine learning model components 400 can be implemented in hardware.

The machine learning model components 400 include a first machine learning model 422, a second machine learning model 424, and an nth machine learning model 426, each of which (individually and/or collectively) can be functionally and/or structurally similar to the machine learning model(s) 224 of FIG. 2, the machine learning model(s) 324 of FIG. 3, and/or the language model(s) 604 of FIG. 6 (described herein). Although three machine learning models are shown in FIG. 4, in some implementations, the machine learning model components 400 can include fewer machine learning models or, alternatively, more machine learning models. For example, the machine learning model components 400 can include a machine learning model for each assessment aspect (e.g., question) of an assessment. The first machine learning model 422 receives input data 402 to produce output data 412, the second machine learning model 424 receives input data 404 to produce output data 414, and the nth machine learning model 422 receives input data 406 to produce output data 416. The input data 402-406 can be functionally and/or structurally similar to the input data 302 of FIG. 3, and the output data 412-416 can be functionally and/or structurally similar to the output data 312 of FIG. 3.

As shown in FIG. 4, the machine learning models 422-426 can be arranged in series, such that data associated with the first machine learning model 422 is provided to the second machine learning model 424, and data associated with the second machine learning model 424 is provided to the nth machine learning model 426. Data is associated with a machine learning model if, for example, the data is included in a context window of that machine learning model. Although not shown in FIG. 4, in some implementations, data associated with a machine learning model can be provided to a plurality of other machine learning models. For example, data from a context window of the first machine learning model 422 can be provided to both a context window of the second machine learning model 425 and a context window of the nth machine learning model 426. Alternatively, data from the context window of the first machine learning model 422 can be provided to the context window of the nth machine learning model 426 and not the context window of the second machine learning model 424.

Each machine learning model from the plurality of machine learning models included in the machine learning model components 400 can be associated with a different assessment aspect (e.g., question). In some implementations, the machine learning model components 400 can have n machine learning models (e.g., language models, multimodal language models, etc.) for n aspects (e.g., questions) of an assessment. For example, for an ALSFRS-R assessment that has 12 aspects (e.g., 12 questions), the machine learning model components 400 can include 12 machine learning models. Alternatively, the machine learning models 400 can include fewer machine learning models than assessment aspects (e.g., such that a machine learning model facilitates multiple aspects of the assessment) or more machine learning models than assessment aspects (e.g., such that multiple machine learning models facilitate a single aspect of the assessment).

To illustrate, first machine learning model 422 can be configured (e.g., prompted) to facilitate a “speech” assessment aspect within an overall ALSFRS-R assessment. Conversation data within the context window of the first machine learning model 422 can be provided to the context window of the second machine learning model 424, which can be configured (e.g., prompted) to facilitate a “salivation” assessment aspect within the overall ALSFRS-R assessment. Conversation data within at least one of the context window of the first machine learning model 422 or the context window of the second machine learning model 424 can be provided to the context window of the nth machine learning model 426, which can be configured (e.g., prompted) to facilitate a “respiratory insufficiency” assessment aspect within the overall ALSFRS-R assessment.

As compared to a single machine learning model, which can, in at least some instances, have reduced accuracy across a plurality of assessment aspects, the machine learning model components 400 having a plurality of machine learning models can have improved accuracy across the plurality of assessment aspects as a result of unique configurations (e.g., unique prompting) across the plurality of machine learning models. The model architecture of the machine learning components 400 can further promote, as compared to a single model, more nuanced and/or detailed interpretations of user inputs and/or more nuanced and/or detailed responses conveyed to the user. Separating models by domain (e.g., assessment aspect) further permits each model to be configured (e.g., prompted) with more complex instructions, such that each model (e.g., agent) can generate more specific follow-up questions, identify user bias more accurately, etc.

As a result of context window data shared among the plurality of machine learning models from the machine learning model components 400, each machine learning model can portray a consistent “personality” to the user. For example, each machine learning model can generate responses that have similar tone, level of detail appropriate for the user's level of familiarity, etc. As a result, the plurality of machine learning models can produce responses that collectively appear to the user to be cohesive and consistent (e.g., as if the responses were produced by a single model). In some instances, each machine learning model from the plurality of machine learning models can be co-trained (e.g., based on a common trained model) such that each machine learning model is associated with a common latent space. As a result, data (e.g., feature vectors) produced by each respective machine learning model can be processed across the plurality of machine learning models.

In some implementations, the machine learning models 422-426 from the machine learning model components 400 can include conversation agents (e.g., agentic models) that can each facilitate a conversation flow. A conversation flow can include a plurality of inputs from the user and a plurality of responses from a conversation agent, before the conversation agent generates an assessment aspect metric value (e.g., a final output for that conversation agent). An example of a conversation flow is described further herein at least in relation to FIGS. 5A-5E.

FIGS. 5A-5E depict an example of a graphical user interface (GUI) 500 configured to facilitate a user assessment, according to an embodiment. The GUI 500 can be implemented and/or displayed by a compute device that is structurally and/or functionally equivalent to the user device 110 of FIG. 1, the compute device 120 of FIG. 1, and/or the compute device 201 of FIG. 2. The GUI 500 can be functionally and/or structurally similar to the user interface 112 of FIG. 1 and/or the user interface 320 of FIG. 3. The GUI 500 portrays first agent data 502 (FIG. 5A), first user input data 504 (FIG. 5A), second agent data 506 (FIG. 5A), speech assessment rubric 508 (FIG. 5A), second user input data 510 (FIG. 5B), third agent data 512 (FIG. 5B), speech assessment metric value indication 514 (FIG. 5B), third user input data 516 (FIG. 5C), fourth agent data 518 (FIG. 5C), salivation assessment metric value indication 520 (FIG. 5C), fifth agent data 522 (FIG. 5D), fourth user input data 524 (FIG. 5D), sixth agent data 526 (FIG. 5D), fifth user input data 528 (FIG. 5D), seventh agent data 530 (FIG. 5E), assessment metric value indications 532 (FIG. 5E), and an overall assessment metric value 534 (FIG. 5E).

The GUI 500 is configured to facilitate an ALSFRS-R assessment. In alternative embodiments not shown in FIGS. 5A-5E, the GUI 500 can be configured to facilitate other assessments, including an ARES assessment, CPIB assessment, a ROADS assessment, a UPDRS assessment, a UHDRS assessment, a CDR assessment, and/or the like. The GUI 500 can portray data generated by a plurality of machine learning models (e.g., that is functionally and/or structurally similar to the machine learning model(s) 324 of FIG. 3 and/or the machine learning models 422, 424, and/or 426 of FIG. 4). A first machine learning model from the plurality of machine learning models can be configured to assess a user's familiarity with the assessment. For example, the first machine learning model can produce the first agent (e.g., assistant) data 502 to ask the user whether the user is familiar with the assessment. Based on the user's response (indicated by the first user input data 504), the plurality of machine learning models can adapt respective outputs to provide a level of detail appropriate for the user's indicated familiarity. The first user input data 504 (and/or at least some of the additional user input data described further below) can include text data (1) typed by a user and/or (2) transcribed from audio data via a microphone (e.g., as described further herein at least in relation to FIG. 6). In some implementations, as described further herein, the first user input data 504 can represent audio data that is input to a machine learning model (e.g., a multimodal LLM), for example, without first being converted to text. In some implementations, the first machine learning model can be different from remaining machine learning models (described below) from the plurality of machine learning models. Alternatively, the first machine learning model can be (or include or be included in) at least one other machine learning model (such as the second, third, and/or fourth machine learning model, described further below) from the plurality of machine learning models.

A second machine learning model from the plurality of machine learning models can be configured (e.g., prompted) to assess a speech aspect of the ALSFRS-R assessment. The second machine learning model can produce the second agent data 506 to ask the user to provide a response for the speech aspect. Concurrent to the display of the second agent data 506, the GUI 500 can also display the speech assessment rubric 508, which can provide definitions for a plurality of assessment metric values for the speech aspect.

In response to viewing the speech assessment rubric 508, the user can input (e.g., via a keyboard, a microphone and a transcription model, etc.) the second user input data 510 to self-define their speech assessment metric value (e.g., a value of 4). In response, the GUI 500 can display a speech assessment metric value indication 514 portraying the user's self-defined speech assessment metric value.

After the user has completed the speech aspect of the assessment, a third machine learning model (e.g., different from the second machine learning model or, alternatively, similar and/or identical to the second machine learning model) can produce the third agent data 512 to guide the user through a salivation aspect of the assessment. The third machine learning model, by producing the third agent data 512, can further confirm the speech assessment metric value as a result of data from the context window of the second machine learning model (e.g., the model associated with the speech assessment aspect) being provided as input to the context window of the third machine learning model. In some implementations, the user can prompt a machine learning model to correct an output (e.g., a generated assessment metric value) of a previous (e.g., upstream) machine learning model. For example, the user can prompt the third machine learning to correct an output of the second machine learning model.

In response to viewing the third agent data 512 relating to the salivation aspect, the user can provide the third user input data 516 to indicate that the user wants further information on the salivation assessment aspect. Based on the user input data 516 indicating that the user has a lower familiarity with the salivation assessment aspect, the third machine learning model can produce the fourth agent data 518 to provide the user with description of metric values for the salivation assessment aspect. The user can then provide input data to select a salivation metric value, which can then be portrayed via the salivation assessment metric value indication 520.

The GUI 500 can further display the fifth agent data 522, which can be produced by a fourth machine learning model (e.g., different from the second and third machine learning models) that is associated with a walk assessment aspect. The fifth agent data 522 can ask the user to assess the user's walking capability. In response to viewing the fifth agent data 522, the user can provide the fourth user input data 524 to request that fourth machine learning model suggest a walking assessment metric value based on the user's condition (e.g., the use of a cane). The fourth machine learning model can then predict a walking assessment metric value based on objective factors (e.g., population data, a user's clinical history data, etc.). This prediction is indicated in the sixth agent data 526 produced by the fourth machine learning model. The user can confirm the predicted walking assessment metric value, as indicated by the fifth user input data 528.

Although some assessment aspects are described herein, in some instances, the GUI 500 can facilitate other assessment aspects. For example, for ALSFRS-R and as shown in FIG. 5, the GUI 500 can collect user-defined metric values for speech, salivation, swallow, write, food, dress, turn, walk, stairs, dyspnea, orthopnea, and respiration. Once the user and/or the assessment application has defined a metric value for each aspect of the assessment (as depicted by the assessment metric value indications 532), the GUI 500 can depict seventh agent data indicating to the user that the assessment is complete. The GUI 500 can further display an indication of the overall assessment metric value 534 (e.g., an ALSFRS-R score), which can include, for example, an aggregate (e.g., a sum, an average, etc.) of the plurality of assessment aspect metric values (e.g., the speech metric value, salivation metric value, etc.).

FIG. 6 is a schematic diagram of a voice processing pipeline 600, according to an embodiment. The voice processing pipeline 600 can be implemented by an assessment system (e.g., that is functionally and/or structurally similar to the assessment system 100 of FIG. 1). The voice processing pipeline 600 can be associated with and/or executed by a compute device (e.g., a compute device that is structurally and/or functionally similar to the compute device 201 of FIG. 2 and/or the user compute device 110 and/or the compute device 120 of FIG. 1). In some instances, for example, the voice processing pipeline 600 can be software stored in memory 210 and configured to be executed via the processor 220 of FIG. 2. In some instances, for example, at least a portion of the voice processing pipeline 600 can be implemented in hardware. The voice processing pipeline 600 includes a speech-to-text model 602, a language model(s) 604 (e.g., that is functionally and/or structurally similar to the machine learning model(s) 324), and a text-to-speech model 606.

The speech-to-text model 602 can receive input audio data (e.g., speech data that is similar to the speech data 304 of FIG. 3) and transcribe the input audio data to produce first text data (e.g., a transcription of uttered words captured in the audio data). The speech-to-text model 602 can include, for example, a transformer-based model. The input audio data can include a recording of a user's speech captured via a microphone. The first text data can be provided as input to the language model(s) 604 (e.g., a transformer-based model, such as a large language model and/or a similar machine learning model configured for text-based natural language processing). As described herein, each language model from the language model(s) 604 can be associated with a different assessment aspect from a plurality of assessment aspects. The language model(s) 604 can produce second text data (e.g., that is responsive to user's statement conveyed in the first text data), which can be provided as input to the text-to speech model 606. The text-to-speech model 606 can include, for example, a transformer-based model, and can be configured to produce audio data that represents/conveys the second text data. An audio signal can be produced based on the audio data, such that the second text data can be audibly communicated to the user via an electroacoustic transducer (e.g., a speaker). In response to hearing the resulting sound, the user can utter an additional statement (e.g., a response) to produce additional input audio data to restart the process implemented by the voice processing pipeline 600.

Some implementations of the voice processing pipeline 600 not shown in FIG. 6 can, for example, exclude at least one of the speech-to-text model 602 and/or the text-to-speech model 606. For example, in some implementations, the language model(s) 604 can include a multi-modal large language model configured to receive as input and/or produce as output audio data (e.g., an encoded audio signal). In some instances, at least a portion of the pipeline can include an API call to a third party computed device (e.g., that hosts a machine learning model).

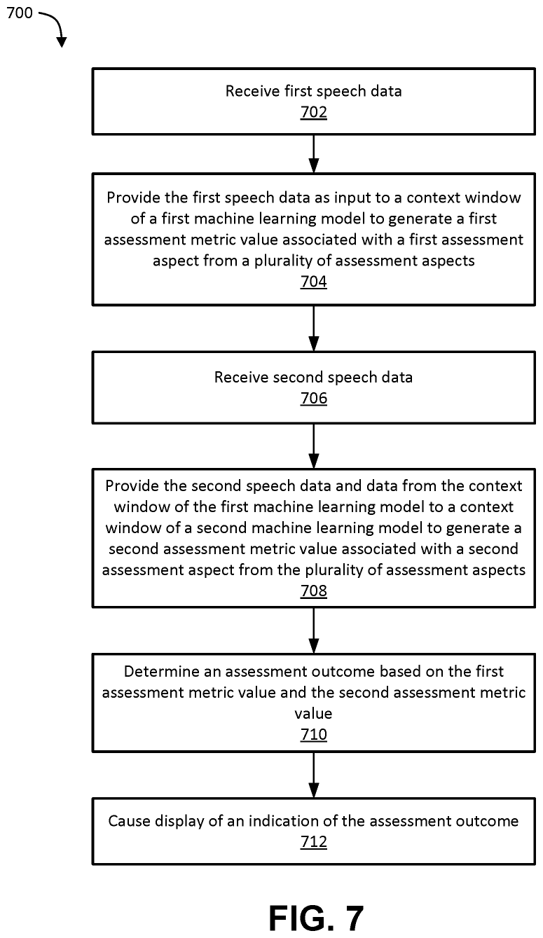

FIG. 7 is a flowchart showing a method 700 of using an assessment system to determine an assessment outcome, according to an embodiment. The method 700 can be implemented by an assessment system described herein (e.g., the assessment system 100 of FIG. 1). Portions of the method 700 can be implemented using a processor (e.g., the processor 220 of FIG. 2) of any suitable compute device (e.g., the compute device 201 of FIG. 2, and/or the user and/or compute devices 110 and/or 120 of FIG. 1).

The method 700, at 702, includes receiving first speech data and, at 704, providing the first speech data as input to a context window of a first machine learning model to generate a first assessment metric value associated with a first assessment aspect from a plurality of assessment aspects. At 706, second speech data is received. The second speech data and data from the context window of the first machine learning model are provided at 708 to a context window of a second machine learning model to generate a second assessment metric value associated with a second assessment aspect from the plurality of assessment aspects. At 710, an assessment outcome is determined based on the first assessment metric value and the second assessment metric value. The method 700 at 712 includes causing the processor to cause display at a user compute device of an indication of the assessment outcome.

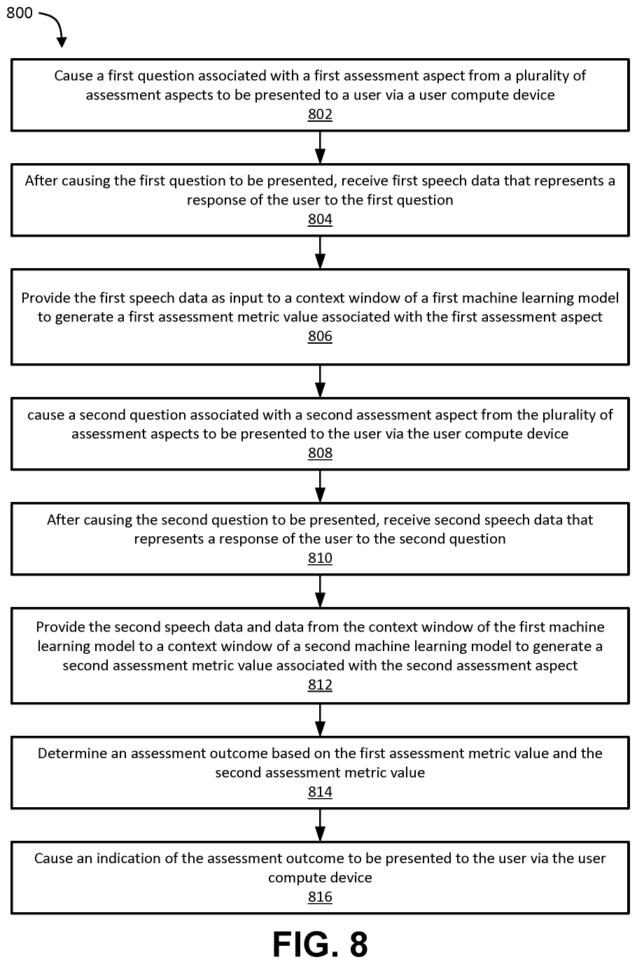

FIG. 8 is a flowchart showing a method 800 for determining an assessment outcome based on a first assessment metric value and a second assessment metric value, according to an embodiment. The method 800 can be implemented by an assessment system described herein (e.g., the assessment system 100 of FIG. 1). Portions of the method 800 can be implemented using a processor (e.g., the processor 220 of FIG. 2) of any suitable compute device (e.g., the compute device 201 of FIG. 2, and/or the user and/or compute devices 110 and/or 120 of FIG. 1).

The method 800 at 802 includes causing a first question associated with a first assessment aspect from a plurality of assessment aspects to be presented to a user via a user compute device. After causing the first question to be presented, the method 800 at 804 includes receiving, at the processor, first speech data that represents a response of the user to the first question. At 806, the method 800 includes providing, via the processor, the first speech data as input to a context window of a first machine learning model to generate a first assessment metric value associated with the first assessment aspect. A second question associated with a second assessment aspect from the plurality of assessment aspects is presented to the user at 808 via the user compute device, and after causing the second question to be presented, the method 800 at 810 includes receiving second speech data that represents a response of the user to the second question. The second speech data and data from the context window of the first machine learning model is provided as input at 812 to a context window of a second machine learning model to generate a second assessment metric value associated with the second assessment aspect. An assessment outcome is determined at 814, based on the first assessment metric value and the second assessment metric value, and an indication of the assessment outcome is presented to the user at 816, via the user compute device.

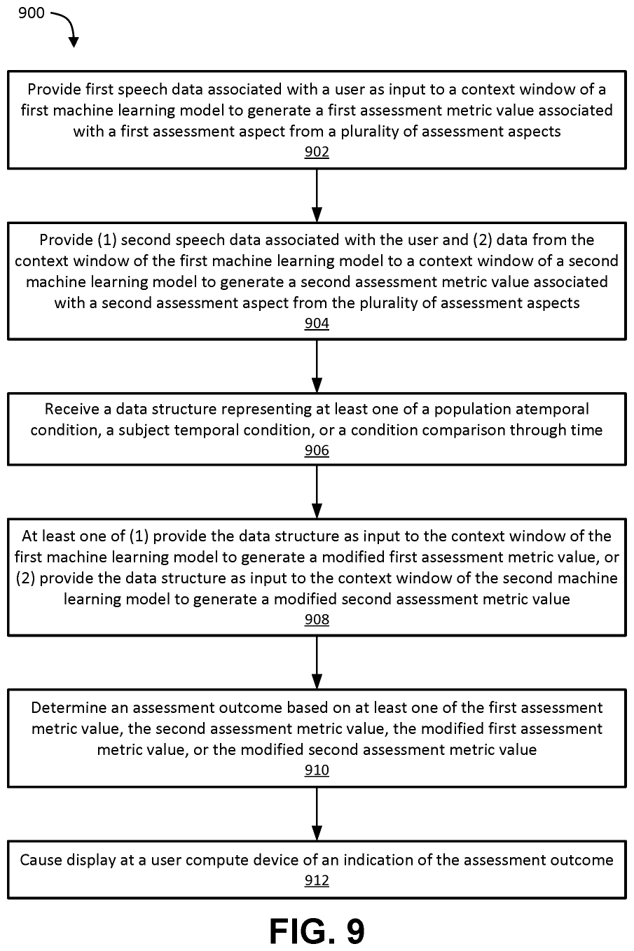

FIG. 9 is a flowchart showing a method 900 for generating modified assessment values, according to an embodiment. The method 900 can be implemented by an assessment system described herein (e.g., the assessment system 100 of FIG. 1). Portions of the method 900 can be implemented using a processor (e.g., the processor 220 of FIG. 2) of any suitable compute device (e.g., the compute device 201 of FIG. 2, and/or the user and/or compute devices 110 and/or 120 of FIG. 1).

The method 900 at 902 includes providing first speech data associated with a user as input to a context window of a first machine learning model to generate a first assessment metric value associated with a first assessment aspect from a plurality of assessment aspects. At 904, the method 900 includes providing (1) second speech data associated with the user and (2) data from the context window of the first machine learning model to a context window of a second machine learning model to generate a second assessment metric value associated with a second assessment aspect from the plurality of assessment aspects. A data structure is received at 906, representing at least one of a population atemporal condition, a subject temporal condition, or a condition comparison through time, and at 908, the method 900 includes at least one of (1) providing the data structure as input to the context window of the first machine learning model to generate a modified first assessment metric value, or (2) providing the data structure as input to the context window of the second machine learning model to generate a modified second assessment metric value. An assessment outcome is determined at 910 based on at least one of the first assessment metric value, the second assessment metric value, the modified first assessment metric value, or the modified second assessment metric value. At 912, the method 900 includes causing display at a user compute device of an indication of the assessment outcome.

Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using Python, Java, JavaScript, C++, and/or other programming languages and development tools. Additional examples of computer code include, but are not limited to, control signals, encrypted code, and compressed code.

The drawings primarily are for illustrative purposes and are not intended to limit the scope of the subject matter described herein. The drawings are not necessarily to scale; in some instances, various aspects of the subject matter disclosed herein can be shown exaggerated or enlarged in the drawings to facilitate an understanding of different features. In the drawings, like reference characters generally refer to like features (e.g., functionally similar and/or structurally similar elements).

The acts performed as part of a disclosed method(s) can be ordered in any suitable way. Accordingly, embodiments can be constructed in which processes or steps are executed in an order different than illustrated, which can include performing some steps or processes simultaneously, even though shown as sequential acts in illustrative embodiments. Put differently, it is to be understood that such features can not necessarily be limited to a particular order of execution, but rather, any number of threads, processes, services, servers, and/or the like that can execute serially, asynchronously, concurrently, in parallel, simultaneously, synchronously, and/or the like in a manner consistent with the disclosure. As such, some of these features can be mutually contradictory, in that they cannot be simultaneously present in a single embodiment. Similarly, some features are applicable to one aspect of the innovations, and inapplicable to others.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. That the upper and lower limits of these smaller ranges can independently be included in the smaller ranges is also encompassed within the disclosure, subject to any specifically excluded limit in the stated range. Where the stated range includes one or both of the limits, ranges excluding either or both of those included limits are also included in the disclosure.

The phrase “and/or,” as used herein in the specification and in the embodiments, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements can optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the embodiments, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the embodiments, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the embodiments, shall have its ordinary meaning as used in the field of patent law.

As used herein in the specification and in the embodiments, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements can optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the embodiments, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) can be those designed and constructed for the specific purpose or purposes. Examples of non-transitory computer-readable media include, but are not limited to, magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices. Other embodiments described herein relate to a computer program product, which can include, for example, the instructions and/or computer code discussed herein.

Some embodiments and/or methods described herein can be performed by software (executed on hardware), hardware, or a combination thereof. Hardware modules can include, for example, a processor, a field programmable gate array (FPGA), and/or an application specific integrated circuit (ASIC). Software modules (executed on hardware) can include instructions stored in a memory that is operably coupled to a processor and can be expressed in a variety of software languages (e.g., computer code), including C, C++, Java™, Ruby, Visual Basic™, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. For example, embodiments can be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

What is claimed is:

1. A non-transitory, processor-readable medium storing instructions that, when executed by a processor, cause the processor to:
receive first speech data;
provide the first speech data as input to a context window of a first machine learning model to generate a first assessment metric value associated with a first assessment aspect from a plurality of assessment aspects;
receive second speech data;
provide the second speech data and data from the context window of the first machine learning model to a context window of a second machine learning model to generate a second assessment metric value associated with a second assessment aspect from the plurality of assessment aspects;
determine an assessment outcome based on the first assessment metric value and the second assessment metric value; and
cause display at a user compute device of an indication of the assessment outcome.

2. The non-transitory, processor-readable medium of claim 1, further storing instructions that cause the processor to:
determine a familiarity metric value based on at least one of a speech tone or a speech pause, represented by the first speech data;
generate response data based on the familiarity metric value; and
cause an audio signal representing the response data to be generated at the user compute device to elicit the second speech data.

3. The non-transitory, processor-readable medium of claim 1, wherein the plurality of assessment aspects is associated with a disease quantification for at least one of amyotrophic lateral sclerosis (ALS), Alzheimer's disease, Huntington's disease, or Parkinson's disease.

4. The non-transitory, processor-readable medium of claim 1, wherein the plurality of assessment aspects is associated with a disease quantification for a neurodegenerative disease.

5. The non-transitory, processor-readable medium of claim 1, further storing instructions to cause the processor to:
receive a data structure representing at least one of a population atemporal condition, a subject temporal condition, or a condition comparison across a period of time; and
at least one of:
provide the data structure as input to the context window of the first machine learning model to generate a modified first assessment metric value, or
provide the data structure as input to the context window of the second machine learning model to generate modified second assessment metric value.

6. The non-transitory, processor-readable medium of claim 1, wherein:
the first machine learning model is a first large language model; and
the second machine learning model is a second large language model different from the first large language model.

7. The non-transitory, processor-readable medium of claim 1, wherein:
the first machine learning model is the second machine learning model.

8. The non-transitory, processor-readable medium of claim 1, further storing instructions to cause the processor to:
receive a first audio signal from the user compute device;
provide the first audio signal as input to a transcription model to produce the first speech data;
receive a second audio signal from the user compute device; and
provide the second audio signal as input to the transcription model to produce the second speech data.

9. The non-transitory, processor-readable medium of claim 1, further storing instructions to cause the processor to:
generate a speech metric value that includes at least one of a speaking rate or an articulation rate, based on at least one of the first speech data or the second speech data;
provide the first speech data and the speech metric value as input to the context window of the first machine learning model to produce a modified first assessment metric value; and
provide the second speech data and the speech metric value as input to the context window of the second machine learning model to produce a modified second assessment metric value, the assessment outcome being determined based on the modified first assessment metric value and the modified second assessment metric value.

10. A method, comprising:
causing, via a processor, a first question associated with a first assessment aspect from a plurality of assessment aspects to be presented to a user via a user compute device;
after causing the first question to be presented, receiving, at the processor, first speech data that represents a response of the user to the first question;
providing, via the processor, the first speech data as input to a context window of a first machine learning model to generate a first assessment metric value associated with the first assessment aspect;
causing, via the processor, a second question associated with a second assessment aspect from the plurality of assessment aspects to be presented to the user via the user compute device;
after causing the second question to be presented, receiving, at the processor, second speech data that represents a response of the user to the second question;
providing, via the processor, the second speech data and data from the context window of the first machine learning model to a context window of a second machine learning model to generate a second assessment metric value associated with the second assessment aspect;
determining, via the processor, an assessment outcome based on the first assessment metric value and the second assessment metric value; and
causing, via the processor, an indication of the assessment outcome to be presented to the user via the user compute device.

11. The method of claim 10, further comprising:
receiving, at the processor, a first audio signal from the user compute device;
providing, via the processor, the first audio signal as input to a transcription model to produce the first speech data;
receiving, at the processor, a second audio signal from the user compute device; and
providing, via the processor, the second audio signal as input to the transcription model to produce the second speech data.

12. The method of claim 10, further comprising:
determining, via the processor, a familiarity metric based on at least one of a speech tone or a speech pause, represented by the first speech data;
generating, via the processor, response data based on the familiarity metric; and
causing, via the processor, the response data to be conveyed at the user compute device to elicit the second speech data.

13. The method of claim 10, wherein the plurality of assessment aspects is associated with a disease quantification for at least one of amyotrophic lateral sclerosis (ALS), Alzheimer's disease, Huntington's disease, or Parkinson's disease.

14. The method of claim 10, wherein the plurality of assessment aspects is associated with a disease quantification for a neurodegenerative disease.

15. The method of claim 10, wherein:
the first machine learning model includes a first agent implemented by a large language model; and
the second machine learning model includes a second agent different from the second agent and implemented by the large language model.

16. A non-transitory, processor-readable medium storing instructions that, when executed by a processor, cause the processor to:
provide first speech data associated with a user as input to a context window of a first machine learning model to generate a first assessment metric value associated with a first assessment aspect from a plurality of assessment aspects;
provide (1) second speech data associated with the user and (2) data from the context window of the first machine learning model to a context window of a second machine learning model to generate a second assessment metric value associated with a second assessment aspect from the plurality of assessment aspects;
receive a data structure representing at least one of a population atemporal condition, a subject temporal condition, or a condition comparison through time;
at least one of:
provide the data structure as input to the context window of the first machine learning model to generate a modified first assessment metric value, or
provide the data structure as input to the context window of the second machine learning model to generate a modified second assessment metric value;
determine an assessment outcome based on at least one of the first assessment metric value, the second assessment metric value, the modified first assessment metric value, or the modified second assessment metric value; and
cause display at a user compute device of an indication of the assessment outcome.

17. The non-transitory, processor-readable medium of claim 16, further storing instructions that cause the processor to:
determine a familiarity metric based on at least one of a speech tone or a speech pause, represented by the first speech data;
generate response data based on the familiarity metric; and
cause the response data to be conveyed at the user compute device to elicit the second speech data.

18. The non-transitory, processor-readable medium of claim 16, wherein the plurality of assessment aspects is associated with a disease quantification for at least one of amyotrophic lateral sclerosis (ALS), Alzheimer's disease, Huntington's disease, or Parkinson's disease.

19. The non-transitory, processor-readable medium of claim 1, wherein the plurality of assessment aspects is associated with a disease quantification for a neurodegenerative disease.

20. The non-transitory, processor-readable medium of claim 1, wherein:
the first machine learning model is a first transformer-based model; and
the second machine learning model is a second transformer-based model different from the first transformer-based model.
